(19) United States
(12) Patent Application Publication
(10) Pub. No.: US 2013/0060143 A1
(43) Pub. Date: Mar. 7, 2013

(54) DOPPLER PROBE PLACEMENT DEVICE AND METHOD OF USE

(71) Applicants: Paulita M. LaPlante, Inver Grove Heights, MN (US); Daniel J. Bartnik, Eden Prairie, MN (US); John Campbell, Eden Prairie, MN (US)

(72) Inventors: Paulita M. LaPlante, Inver Grove Heights, MN (US); Daniel J. Bartnik, Eden Prairie, MN (US); John Campbell, Eden Prairie, MN (US)

(73) Assignee: Vasamed, Inc., Eden Prairie, MN (US)

(21) Appl. No.: 13/599,523

(22) Filed: Aug. 30, 2012

Related U.S. Application Data
(60) Provisional application No. 61/531,877, filed on Sep. 7, 2011.

(51) Int. Cl. A61B 8/08

(52) U.S. Cl. 600/453

(57) ABSTRACT

The present invention is directed towards a placement device for a flat Doppler probe and a method of use that allows for hands-free use of a flat Doppler probe. The device replaces gel for transmitting and receiving ultrasound signals or alternatively encapsulates the gel. The placement device includes a hydrophilic surface that contacts a patient and allows for the transmission and receiving of ultrasound signals. The placement device eliminates the use of gel for easier clean up and optimizes the transmission of ultrasound signals. The placement device includes an adhesive on the patient contacting surface that allows the placement device to adhere to a surface on a patient.

This application is a non-provisional patent application of U.S. application Ser. No. 61/531,877, filed on Sep. 7, 2011, the entirety of which is hereby incorporated by reference.

### FIELD OF THE INVENTION

The present invention is directed to a Doppler probe placement device for use with flat Doppler probes. In particular, the Doppler probe placement device minimizes operator manipulation and maximizes conduction of the probe.

### BACKGROUND OF THE INVENTION

Ultrasound scanning or diagnostic sonography, as it is known in the medical field, is a technology that uses cyclic sound pressure at a frequency greater than what humans can hear to image or view soft tissues in the human body. Typically sonography is more effective imaging organs that are soft or filled with fluid rather than bone or organs filled with air. Sonography can capture images of the body or provide real time tomographic images. It is one of the most widely used imaging technologies in the medical field due to it being relatively inexpensive and portable, especially when compared to other techniques such as magnetic resonance imaging (MRI) and computed tomography (CT). Another benefit of sonography is that it is a “safe test” because it does not use ionizing radiation.

There are many uses of sonography in the medical field such as obstetric diagnostics, scanning organs and musculo-skeletal imaging.

Sonography emits and detects high frequency waves that are measured by a computer and translated into an image. Typically this is done by an ultrasound probe used in combination with a small amount of transmission material such as a gel. The gel is placed on the patient's skin over the area to be scanned. The probe is then manually slid over the area where the transducer emits the sound waves that penetrate the patient's body and the waves echo from objects within the patient's body such as organs and bones. The transducer receives the reflected waves and a computer processes them to be viewable as an image.

A common use of sonography is Doppler ultrasound to non-invasively measure the velocity and direction of moving structures such as blood flow. The Doppler probe itself is traditionally a pencil configuration with a rounded or flat tip. These probes must be held at an angle to blood flow in order to accurately capture lower extremity blood flow. A flat Doppler probe is typically used when a signal is required for an extended or prolonged period of time. In these cases however, the clinician or technician may be unable or unwilling to position the probe at the correct angle for the duration of the test. In addition, it is more difficult to obtain Doppler signals on patients with peripheral arterial disease because of the compromised blood flow in the lower extremities. Thus, a great deal of skill is required to successfully identify and measure arterial flow in these patients.

Another drawback of conventional Doppler probes is that a liquid gel is used to optimize the transmission of the Doppler signal through skin and it is difficult to keep the gel in place. It is also difficult to dispense the correct amount of gel. Too much or too little affects the quality of the Doppler signal. Furthermore, the gel must be cleaned from skin and instrument surfaces, and gel must also be cleaned from the Doppler probe itself.

U.S. Pat. App. Pub. No. 2011/0015527 to Heasty et al. attempts to address some of the foregoing problems by disclosing a flat Doppler probe having up to two crystals, one for transmitting Doppler signals and the other for receiving the signals. The Doppler probe is designed to have the Doppler crystal at an optimal angle for ease of use in order to accurately capture lower extremity blood flow and a recess in the flat surfaces to allow for enough gel to aid in receiving the Doppler signals. While the Heasty et al. probe is an advance over conventional flat Doppler probes, it does not alleviate the problem of holding the Doppler probe for extended periods of time, finding the optimal signal from blood flow, holding the Doppler probe steady during cuff inflation and deflation, which may be manual or automated, and in addition managing data collection. In addition, while the Heasty et al. probe makes it easier to clean the gel out of the recession area, the gel still needs to be cleaned out of the probe and from the patient, which is time consuming for the clinician and/or technician. Moreover, the Heasty et al. probe does not solve the problem associated with reproducible data collection when multiple readings throughout a day are required.

Accordingly, there is a need and desire to provide a system and method for allowing the hands free operation of a flat Doppler probe that eliminates the need for ultrasound gel and assures reproducibility of data.

### BRIEF SUMMARY OF THE INVENTION

The present invention addresses the problems outlined above by providing a probe placement device that permits a flat Doppler probe to be securely but removably positioned on a patient and maximizes conduction of the probe by providing an optimal amount of transmission material therewithin.

Embodiments of the present invention advantageously provide a Doppler probe placement device that replaces the use of gel as a conducting material during an ultrasound or Doppler sonography. The placement device includes top and bottom surfaces with first, second, third and fourth sides defining a probe receiving chamber therewithin. At least one of the sides includes a probe receiving opening to receive a flat Doppler probe into the receiving chamber. The bottom surface contacts the patient and comprises a material that allows for the transmission of Doppler signals. The material may be hydrophilic in nature. The top surface may be made of the same transmissive material or may be a non-transmissive material. Alternatively, the placement device may enclosedly contain an effective, optimal volume of ultra-sound gel. Thus, by enclosing the ultra-sound gel within the placement device itself or by eliminating the use of ultrasound gel altogether, the flat Doppler probe placement device reduces cleanup and obtains an optimal signal to and from the probe.

In another embodiment of the invention the flat Doppler probe placement device includes an adhesive coating around a perimeter of the contacting surface. The contacting surface may also include a removable protective covering that protects the adhesive during storage, transportation and other non-use situations until the placement device is ready to be positioned on a patient. The adhesive coating allows for hands free operation of the flat Doppler probe.

In another embodiment of the invention a method of obtaining a Doppler reading is disclosed. The clinician or technician inserts the flat Doppler probe into the probe receiving opening of the placement device and locates the optimal placement of the flat Doppler probe on the patient. After locating the optimal placement of the probe, the clinician or technician removes the protective covering exposing the adhesive and then securely places the placement device including the Doppler probe therewithin on a patient. The clinician or technician then transmits and receives reflected Doppler signals with the flat Doppler probe. The signal is then processed by a computer to obtain a Doppler spectrum.

### BRIEF DESCRIPTION OF THE DRAWINGS

FIG. 1 is a side view of the flat Doppler probe placement device in accordance with the invention showing a flat Doppler probe therewithin.

FIG. 2 is a top plan view of the flat Doppler probe placement device in accordance with the invention showing a flat Doppler probe therewithin.

### DETAILED DESCRIPTION OF THE INVENTION

The present invention is directed to a placement device for flat Doppler probes to aid in transmitting and receiving Doppler signals. The present invention also discloses a method of using the placement device to obtain a Doppler spectrum.

It should be noted that this is an exemplary embodiment and use of the present invention. The apparatus and method may be used outside the treatment and diagnosis of humans. For example it may be used on animals. Furthermore, there may be other variations of the placement device that will be appreciated by one skilled in the art that relate to flat Doppler probes or other ultrasound devices.

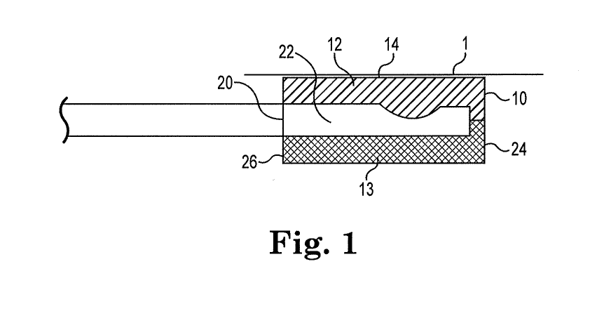

FIG. 1 is a side view of an exemplary embodiment of the flat Doppler probe placement device 10 in accordance with the invention. The placement device 10 includes bottom 12 and top 13 surfaces and distal 24 and proximal 26 ends. The bottom surface 12 contacts a surface 1 of a patient, such as the skin. The bottom surface 12, or the patient contacting surface, may comprise a material suitable for transmitting and receiving Doppler signals. In one embodiment, the material may be a hydrophilic material that is optimized to conduct ultrasound signals thus eliminating the need for using ultrasound gel. The hydrophilic material should be capable of transmitting a frequency in the range of 5 to 8 mHz. Those of skill in the art will appreciate that suitable materials include hydrophilic materials having low tear strength such as HEMA:VP (hydroxyethyl methacrylate vinyl pyrrolidone) or MMA:VP (methyl methacrylate vinyl pyrrolidone)), as well as hydrophilic materials having relatively high tear strength such as AN:VP (acrylonitrile vinyl pyrrolidone), HEMA:MMA (hydroxyethyl methacrylate methyl methacrylate) or polyamide vinyl pyrrolidone. The material may also be of optimal thinness and durometer to minimize interference with Doppler transmitting and receiving signals. The top surface 13 of the flat Doppler probe placement device 10 in accordance with the invention opposes the bottom surface and is securedly coupled thereto. Those of skill in the art will appreciate that the two surfaces 12, 13 may be secured by thermal bonding, adhesive, or the like. The top surface may comprise the same material as the patient contacting surface 12 or alternatively may comprise a material that is non-transmissive or in other words not suitable for transmitting and receiving Doppler signals. Ideally, the top and bottom surfaces are made from clear film to allow the clinician to view the placement of the Doppler probe.

In an alternative embodiment (not shown), the placement device 10 may include two bottom surfaces that are thermally or adhesively bonded together and enclose an optimal volume of ultrasound gel. One bottom surface is the patient contacting surface while the “inner” bottom surface bonds with the top surface. In this way, ultrasound gel is used but is conveniently contained within the placement device 10.

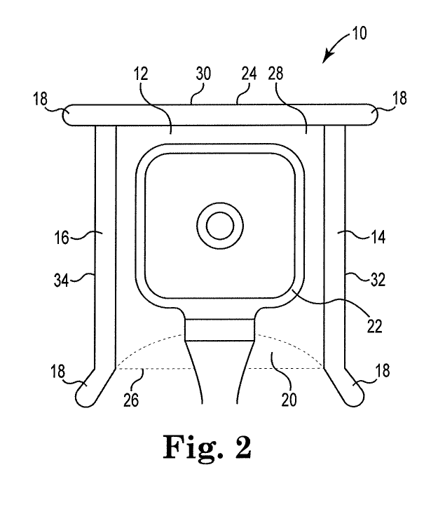

The flat Doppler probe placement device 10 in accordance with the invention may be treated with an adhesive coating 14 on the patient contacting surface or around the perimeter of patient contacting surface 12. Preferably, the adhesive is placed around the perimeter of the patient contacting surface 12 to ensure that there is no interference with the Doppler signals. The adhesive coating 14 may include any repositionable, pressure sensitive adhesive that does not interrupt physiological parameter monitoring. The adhesive coating is an inherently tacky, elastomeric, solvent-dispersible, solvent-insoluble pressure sensitive adhesive. In one embodiment, the adhesive coating is a monomer or polymer blend selected from the group consisting of alkyl acrylate, alkyl methacrylate ester, acrylic acid, methacrylic acid, itaconic acid, crotonic acid, maleic acid, fumaric acid, sulfoethyl methacrylate, and ionic monomers such as sodium methacryate, ammonium acrylate, sodium acrylate, trimethylamine p-vinyl benzimide, 4,4,9-trimethyl-4-azonia-7-oxo-8-oxa-dec-9-ene-1-sulphonate, N,N-dimethyl-N-(.beta.-methacryloxyethyloxy-ethyl) ammonium propionate betaine, trimethylamine methacrylimide, and 1,1-dimethyl-1-(2,3-dihydroxypropyl)amine methacrylimide. In another embodiment, the adhesive coating may include microspheres selected from the group consisting of acrylate, alkylacrylate and alkylacrylate ester monomers alone or in combination with vinyl monomers. The adhesive coating may be covered with a removable paper or other type of film used to maintain the tackiness of the adhesive coating during storage, transportation and other non-use situations. As best seen in FIG. 2, the removable cover may also include tab portions 18 that extend past the perimeter of the placement device 10, allowing the user to easily remove cover 16 to expose the adhesive 14. When the placement device is adhesively placed in position on the patient, it allows the “hands-free” operation of the Doppler probe by the clinician or technician.

Those of skill in the art will appreciate that the size and shape of the placement device 10 can be varied depending on the size and shape of the flat Doppler probe with which it is used.

Referring now to FIG. 2, a plan view of the flat Doppler probe placement device 10 is shown. Proximal end 26 of the placement device 10 includes receiving opening 20 while distal end 24 is secured to top and bottom surfaces as previously described. First, second and third sidewalls 30, 32, 34 define a probe receiving chamber 28 therewithin. As depicted, receiving opening 20 is arcuate shaped. However, those of ordinary skill in the art will appreciate the receiving opening 20 may be sized or shaped to receive any size or shape of flat Doppler probe. Flat Doppler probe 22 is inserted through receiving opening 20 and positioned within receiving chamber 28 of the probe placement device 10. The removable cover may optionally include tab portions 18 that extend past the perimeter of the placement device 10, allowing the user to easily remove cover 16 to expose the adhesive 14.

In operation, a clinician or technician positions the placement device 10 through arcuate receiving opening 20 and determines the correct placement of the device on a surface of a patient. The clinician views the placement of the device through the clear top and bottom surfaces and slides the flat Doppler probe within the placement device 10 over the skin surface. When the ideal surface is located, the clinician or technician grasps tab portions 18 and easily removes cover 16, exposing the adhesive 14. The placement device 10 containing the flat Doppler probe is then pressed on the skin causing the adhesive to secure the placement device 10 to the patient. The flat Doppler probe 22 then transmits and receives reflected Doppler signals, which are then processed by computer means to obtain a Doppler spectrum.

Although the description of the preferred embodiment has been presented, it is contemplated that various changes, including those mentioned above, could be made without deviating from the spirit of the present invention. It is therefore desired that the present embodiment be considered in all respects as illustrative, not restrictive, and that reference be made to the appended claims rather than to the foregoing description to indicate the scope of the invention.

What is claimed is:

1. A Doppler probe placement device comprising:
a patient contacting surface comprising a material suitable for transmitting and receiving Doppler signals;
a top surface opposing said patient contacting surface and operably coupled thereto to form first, second, third and fourth sides defining a probe receiving chamber therewithin; and
a receiving opening on at least one of said first, second, third or fourth sides for receiving a Doppler probe.

2. The placement device according to claim 1 wherein said material has a thickness and durometer that minimizes interference with Doppler transmitting and receiving signals.

3. The placement device according to claim 2 wherein said material is hydrophilic.

4. The placement device according to claim 1, further comprising:
an adhesive coating on the patient contacting surface that is configured to secure said placement device on the surface of a patient.

5. The placement device according to claim 4, wherein said adhesive is located around a perimeter of the patient contacting surface.

6. The placement device according to claim 4 further comprising:
a removable cover for protecting the adhesive before use.

7. The placement device according to claim 1 wherein said device is disposable.

8. The placement device according to claim 1 wherein said device is reusable.

9. The placement device according to claim 1 wherein said patient contacting surface includes a top layer secured thereto and defining an ultrasound gel cavity therewithin, said ultrasound gel cavity including an effective volume of ultrasound gel.
